(19) United States
(12) Patent Application Publication
(10) Pub. No.: US 2022/0047744 A1
(43) Pub. Date: Feb. 17, 2022

(54) CHARGING ASSEMBLY FOR SANITIZING CONTAINER

(71) Applicant: Santrail, LLC, Mobile, AL (US)

(72) Inventors: Sheffield Clark, Southlake, TX (US); Craig Zieres, Fort Worth, TX (US)

(73) Assignee: Santrail, LLC, Mobile, AL (US)

(21) Appl. No.: 17/404,940

(22) Filed: Aug. 17, 2021

Related U.S. Application Data
(60) Provisional application No. 63/066,636, filed on Aug. 17, 2020.

(51) Int. Cl. A61L 2/10; A61L 2/26; H02J 7/00

(52) U.S. Cl. CPC A61L 2/10; A61L 2/26; A61L 2202/23; A61L 2202/11; A61L 2202/16; H02J 7/0044

(57) ABSTRACT

A system and method for a charging assembly designed to sanitize the exterior of sanitizing containers is provided. The system and method of the present disclosure are generally designed to provide power to a plurality of sanitizing containers within a cavity that contains ultraviolet (UV) emitters used to sanitize the exterior of the sanitizing containers. To this end, the system and method of the present disclosure may comprise a vessel, charging pins, power supply, and circuitry connecting the power supply to the various components of the system. Spacing rods may be used to support the containers within the internal cavity at an angle to reduce movement, which may allow for a more even distribution of UV light. To reduce unwanted exposure to UV light, switches may be used to allow for the control of power to the UV emitters.

### CROSS REFERENCES

This application claims the benefit of U.S. Provisional Application No. 63/066,636, filed on Aug. 17, 2020, which application is incorporated herein by reference.

### FIELD OF THE DISCLOSURE

The subject matter of the present disclosure refers generally to a system and method for a charging assembly of a sanitizing container.

### BACKGROUND

Fomites, such as currency, can carry viruses, protozoa, and bacteria, and have been found to carry pathogens ranging from lethal viruses, such as COVID-19, to skin irritating bacteria responsible for acne, such as propionibacterium acnes. A person can easily spread these pathogens to others by touching infected currency prior to physically contacting another person. In some instances, a person may unintentionally spread a pathogen to others by touching currency prior to touching some other fomite, such as a door handle, meaning pathogens on currency can be spread to others without even requiring person to person contact. This is particularly a problem for those who work at banks or manage ATMs since these people are at a higher risk of being exposed due to the large amount of currency handled on any given day. Additionally, security measures necessary to protect currency can make it difficult for the incorporation of sanitation procedures in an efficient manner.

Though currency often sits in buildings and transport vehicles without undergoing any type of sanitation treatment, it is inefficient to require someone to collect the currency and then transfer the currency to a sanitation device prior to transporting the currency to its finally destination. This would also expose those who collect the currency prior to sanitation to pathogens. Optimally, a container that uses heat and UV light may be used to sanitize currency; however, currently there are no containers that sanitize currency and other fomites using these methods. And even if there were containers that were optimized to sanitize via heat and UV radiation, there is currently no solution for providing power to these containers while being transported from one place to another. Additionally, even if the contents within the containers was sanitized, the exterior surfaces of the containers themselves may be carriers of dangerous pathogens for which the containers were designed to kill.

Therefore, there is a need in the art for a charging assembly that can both provide power to containers used to sanitize currency while also sanitizing the exterior surface of the containers.

### SUMMARY

A system and method for sanitizing containers is provided. In one aspect, the system allows users to sanitize containers configured to hold currency by placing said container in an internal cavity containing UV emitters configured to emit at wavelengths sufficient to kill organisms that cause disease. In another aspect, the system provides power to containers so that they may undergo their own sanitation process. Generally, the system allows users to sanitize currency and containers in which said currency is contained. The system generally comprises a vessel, charging pins, power supply, and circuitry connecting the power supply to the various components of the system. Spacing rods may be used to support the containers within the internal cavity at an angle to reduce movement, which may allow for a more even distribution of UV light. To reduce unwanted exposure to UV light, switches may be used to allow for the control of power to the UV emitters. Some embodiments may comprise a locking device located on the bottom of the vessel, which may allow the charging assembly to be secured to a stationary fastener of a vehicle.

The foregoing summary has outlined some features of the system and method of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the system and method disclosed herein. Those skilled in the pertinent art should also realize that such equivalent designs or modifications do not depart from the scope of the system and method of the present disclosure.

### DESCRIPTION OF THE DRAWINGS

These and other features, aspects, and advantages of the present disclosure will become better understood with regard to the following description, appended claims, and accompanying drawings where:

FIG. 1 is a diagram illustrating a charging assembly embodying features consistent with the principles of the present disclosure.

FIG. 2 is a diagram illustrating a charging assembly embodying features consistent with the principles of the present disclosure.

FIG. 3 is a diagram illustrating a charging assembly embodying features consistent with the principles of the present disclosure.

FIG. 4 is a diagram illustrating a charging assembly embodying features consistent with the principles of the present disclosure.

FIG. 5 is a diagram illustrating a charging assembly embodying features consistent with the principles of the present disclosure.

FIG. 6 is a diagram illustrating a charging assembly embodying features consistent with the principles of the present disclosure.

FIG. 7 is a flow chart illustrating certain method steps of a method embodying features consistent with the principles of the present disclosure.

### DETAILED DESCRIPTION

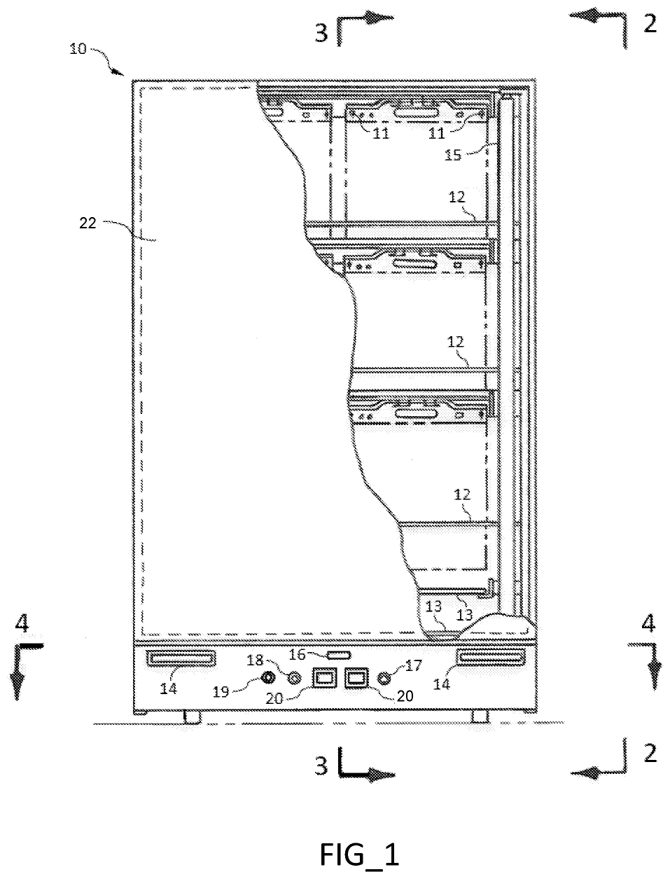

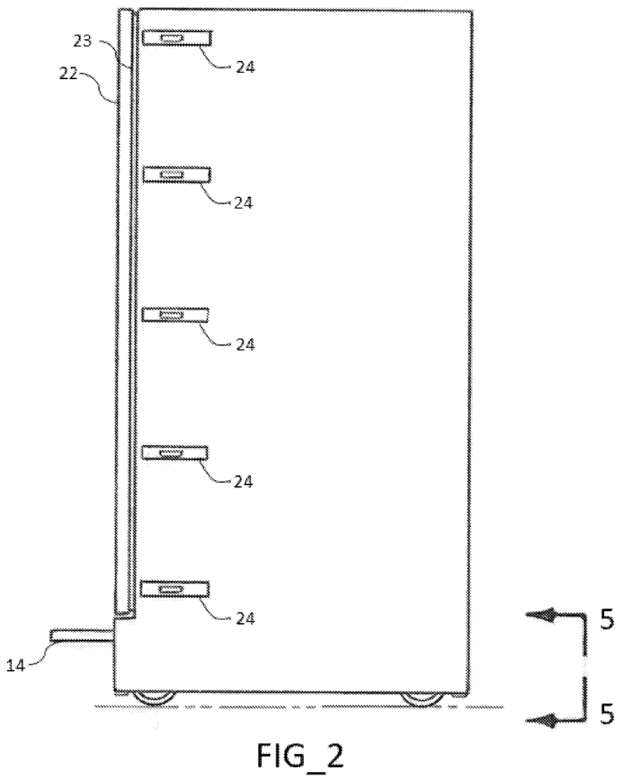

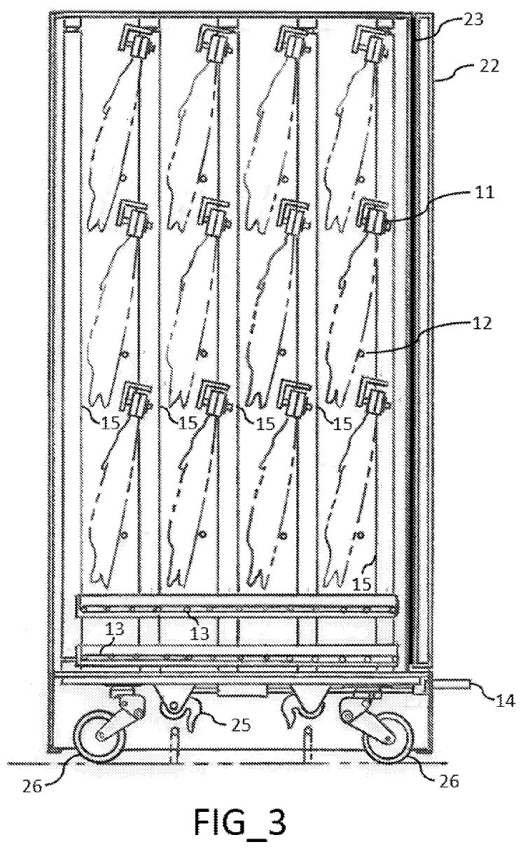

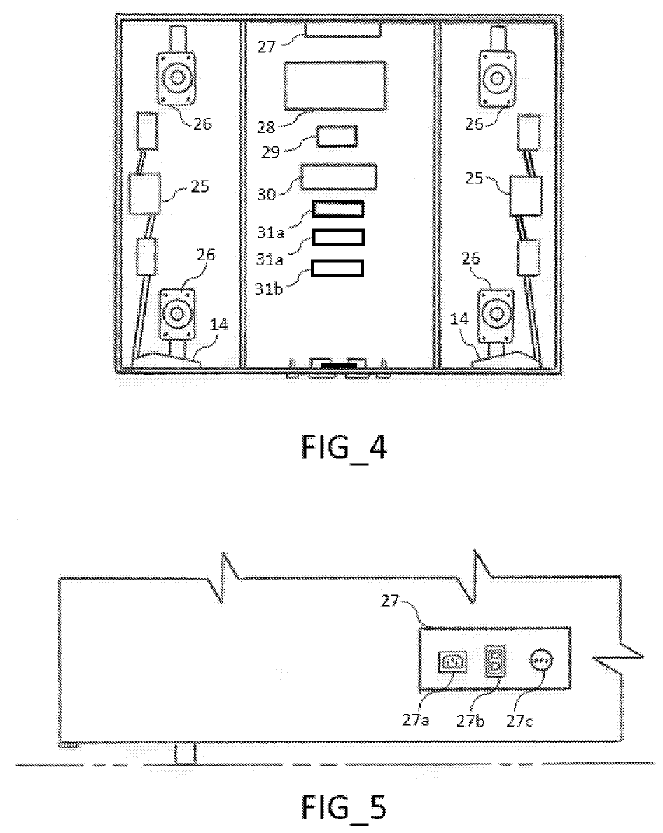

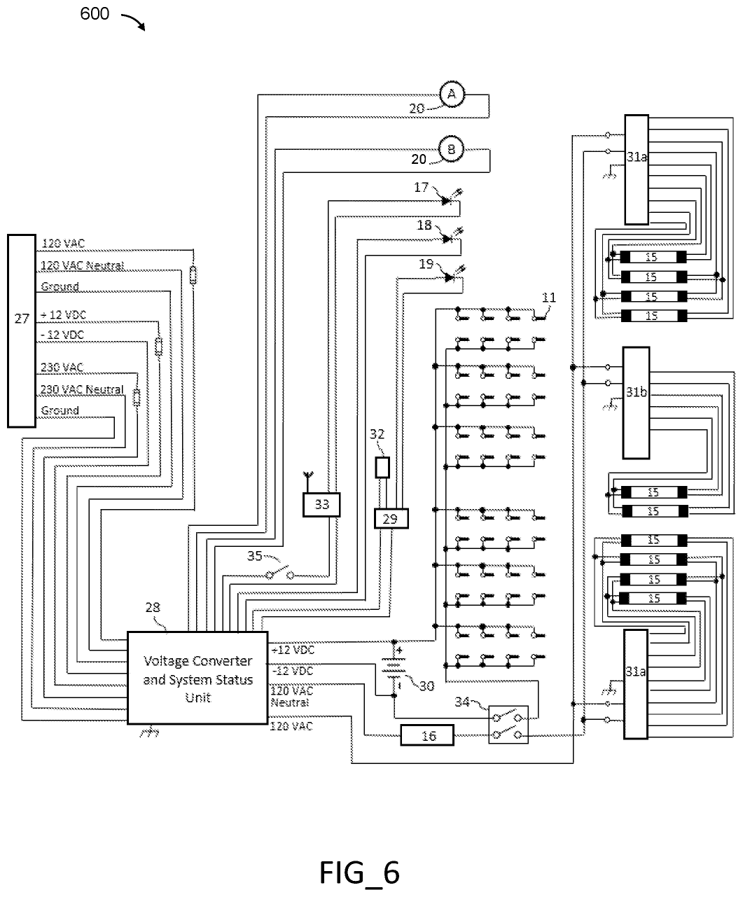

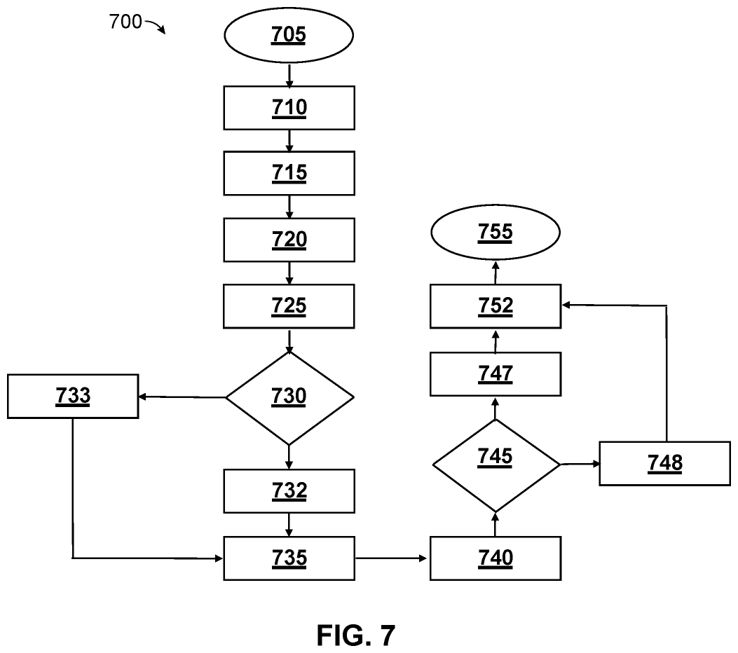

A system and method for a charging assembly for a sanitizing container is provided. Generally, the system 100 provides power to a plurality of sanitizing containers within a cavity that contains ultraviolet (UV) emitters used to sanitize the sanitizing containers. FIGS. 1-6 illustrate embodiments of a charging assembly used to charge sanitizing containers. FIG. 1 depicts a front sectional view of a preferred embodiment of a charging assembly, including UV emitters 15 used to clean the sanitizing containers of the system 100. FIG. 2 depicts a side view of a preferred embodiment of a charging assembly. FIG. 3 depicts a preferred embodiment of the interior of a charging assembly containing a plurality of sanitizing containers. FIG. 4 depicts a plan view of the preferred embodiment of the base of a charging assembly. FIG. 5 depicts a plurality of power supplies that may be used to power the system 100. FIG. 6 depicts the circuitry 600 of the system 100, including a control board and at least one switch 34, 35 that may be used to turn the UV lights on and off as well as to control the power transferred to the sanitizing containers. FIG. 7 illustrates a method that may be carried out by a user using the charging assembly. It is understood that the various method steps associated with the methods of the present disclosure may be carried out using the sanitizing container depicted in FIGS. 1-6.

As illustrated in FIG. 1, the charging assembly generally comprises a vessel 10, charging pins 11, power supply 30, and circuitry 600 connecting the power supply 30 to the various components of the system 100. Other preferred embodiments may further comprise a control board, at least one LED and/or a UV emitting device, at least one switch 34, 35 within the circuitry 600, and spacing rods 12 that may be used to space the contents within the internal cavity of the charging assembly. Generally, currency sanitizing containers are connected to the charging pins 11 at charging ports. This may provide the currency sanitizing containers with power so that heating elements of the currency sanitizing containers may heat currency within to a temperature known to kill pathogens. The control board of the currency sanitizing containers regulates the flow of power to the heating elements from the power supply 30 of the sanitizing container to maintain the desired temperature until a desired period of time has elapsed. UV emitters 15 within the internal cavity of the charging assembly may sanitize the exterior surface of the sanitizing containers. In one preferred embodiment, power transferred to the UV emitters 15 and charging pins 11 from the power supply 30 may be stopped via an interlock switch 34 connected to the door 22 of the vessel 10. When a door 22 of the vessel 10 is in an open position, the interlock switch 34 may break the circuit and when the door 22 is in a closed position, the interlock switch 34 may close the circuit. This may prevent users of the device from being shocked by the charging pins 11 or exposed to UV radiation from the UV emitters 15.

The vessel 10 holds the sanitizing containers and houses the various components of the system 100. In a preferred embodiment, the vessel 10 is a rigid box and comprises an internal cavity created by at least two sides, top, bottom, and door 22. In some preferred embodiments, the bottom and/or top of the device may have a volume in which components of the system 100 may be stored, as illustrated in FIG. 4. These components may include, but are not limited to a power entry module, transformer 28, system status unit, UV intensity monitor, and power supply 30. Ballasts 31a, 31b for the charge rails and/or UV lights of the system 100 may also be attached within the volume of the bottom of the vessel 10, which may allow circuitry 600 to supply power to the charging pins 11 and UV emitters 15. Types of materials that may be used to construct the container include, but are not limited to, steel, aluminum, polymer, or any combination thereof. In one preferred embodiment, the container may comprise a flame retardant and/or insulating material lining the interior surface and/or exterior surface of the vessel 10 in case of a fire or electrical short. For instance, the vessel 10 may comprise a steel shell lined with mineral wool on the exterior surface, wherein the mineral wool is bonded to the exterior surface in a way such that the mineral wool may prevent the transfer of heat to the exterior of the vessel 10 should a sanitizing container have a short and catch fire. As illustrated in FIG. 2, a hook latch 24 may be used to hold the door 22 in place. In a preferred embodiment, the hook latch 24 may be used to hold the door 22 tight against a seal 23 in order to tightly seal the internal cavity of the charging assembly. In a preferred embodiment, the seal is a bulb seal 23, as illustrated in FIG. 2.

FIG. 3 illustrates a preferred means in which sanitizing containers are positioned within the charging assembly. In a preferred embodiment, the charging pins 11 are located on a plurality of parallel charging rails within the internal cavity of the vessel 10 and are spaced to allow charging ports of multiple sanitation containers to be attached thereto. Spacing rods 12 may be positioned so that they contact the bottom half of any sanitizing containers attached to the spacing rod's 12 corresponding charging rail. As illustrated in FIG. 3, the spacing rod 12 is not vertically aligned with its corresponding charging rail, which may cause the sanitizing container to not form a ninety-degree angle with the bottom of the container. Offsetting the spacing rods 12 in the manner illustrated in FIG. 3 reduces the amount of movement the sanitizing containers experience within the vessel 10 when moved. UV emitters 15 are preferably positioned within the cavity to maximize the amount of UV radiation within the inner cavity. In one preferred embodiment, the UV emitters 15 may be attached to the charging rails opposite the charging pins 11. In another embodiment, the UV emitters 15 may be attached to the interior walls of the vessel 10. In yet another preferred embodiment, a lighting structure may be used to suspend the UV emitters 15 throughout the internal cavity of the vessel 10. Wire racks 13 may be used to hold items other than sanitizing containers within the cavity, allowing the UV emitters 15 of the device to sanitize their exterior surfaces as well.

In one preferred embodiment, wheels 26 may be attached to the vessel 10, as illustrated in FIG. 1-4. Wheels 26 may allow a user to transport the system 100 without the need of a vehicle. In a preferred embodiment, the system 100 may comprise spring-loaded wheels 26 configured to compress when a downward force is applied thereto. This may allow the vessel 10 of the charging assembly to rest on the floor of a transport vehicle to increase friction and prevent movement. It may also allow a charging assembly to be secured to the floor of a bank or inside a vault, thus making it more difficult to steal. In one preferred embodiment, the bottom of the vessel 10 may have a high friction material, such as rubber, attached to its exterior surface to further increase friction and prevent movement. When the wheels 26 are compressed, the high friction material of the vessel 10 may help hold the system 100 in place. The material may also be used to attenuate vibrations that may occur when the system 100 is secured to a moving vehicle.

The downward force may be applied to the system 100 via a locking device of the vessel 10, as illustrated in FIGS. 3 and 4. The locking device may comprise a hook, tow, and engine. The engine preferably comprises a motor, gear train, and drum. In a preferred embodiment, the motor is an electric motor having an armature and a stator. The armature may be defined as rotor having a magnetic field that may be used to generate torque, and the stator may be defined as an outer set of permanent magnets or field coils that interact with the magnetic field of the armature to generate torque. By altering the magnetic field of the stator, the armature rotates in a way that produces the aforementioned torque. In some preferred embodiments, multiple engines may be attached to the vessel 10 so that they may work in tandem to apply a downward force to the charging assembly. At least one tow in the form of a cable is preferably attached to the drum of the engine in a way such that it may wind around the drum. The motor may turn the drum about a central axis, which may cause the cable to spool or unspool from the drum. The cables are preferably attached to a locking element 25, such as a hook, at one end, which may allow the engine manipulate the manipulate the locking element 25 via the cables. In one preferred embodiment, the tow in which the locking element 25 may be attached is a stationary fastener on a base surface, which may allow the locking device to pull the system 100 downward as the cable is spooled about the drum. For instance, a system 100 having a hook may attach to stationary fasteners of a vehicle's floor in order to keep the vessel stationary while said vehicle is in motion. As the system 100 spools the cable around the drum, the hook holds onto the stationary fastener of the floor, thus causing the motor to pull the system 100 downward toward the floor as the cable continues to spool about the drum. In one preferred embodiment, the cable may be spooled until the base of the vessel 10 contacts the floor of the vehicle, at which point the engine would shutoff. The locking device is preferably contained within the bottom of the system 100, as illustrated in FIG. 4.

In another preferred embodiment, the engine may comprise a motor and a worm gearbox having a worm shaft, worm gear, and worm gear shaft. The worm shaft comprises threads that engage the worm gear in a way such that when the worm shaft is rotated, the worm gear rotates perpendicular relative to the worm shaft. The worm gear shaft is connected to the worm gear so that it rotates as the worm gear rotates. In a preferred embodiment, the worm shaft is operably connected to the motor in a way such that it may turn the worm shaft about said central axis, which in turn causes the worm gear shaft to rotate. A locking element 25 attached to said worm gear shaft may engage the tow as the worm gear shaft is rotated, thus locking the system 100 in place. By reversing rotation of the engine, a user may cause the locking element 25 to disengage the tow, which unlocks the system 100 from a stationary position. In yet another embodiment, as illustrated in FIG. 2, a latch 14 may be used to further secure the system 100 during transport. In one preferred embodiment, the latch 14 is a tow hitch that may be used to move the system 100 from one location to another with the assistance of a vehicle fitted with a ball that fits said tow hitch. This may allow users to more easily transport the system 100 when it is loaded with sanitizing containers.

The power supply 30 may be connected to the circuitry 600 of the system 100 in a way such that the charging pins 11 and UV emitters 15 may receive power from the power supply 30, thus allowing the sanitizing containers to receive power via the charging pins 11 and sanitizing UV radiation from the UV emitters 15. Types of power supplies that may be used to supply the charging pins 11 and UV emitters 15 with power include, but are not limited to, batteries, wall outlets, alternators, or any combination thereof. In the preferred embodiment as illustrated in FIG. 4, the power supply 30 is a battery. In some preferred embodiments, the batteries may be removable so that a user may easily replace them. The batteries may be on board the vessel 10 or part of another device operably connected to the system 100. For instance, an armored truck having an alternator may be configured to provide power to the system 100 via an alternator outlet plug connected to the power entry panel 27 of the vessel 10, which may comprise various receptacles, including but not limited to, 120 VAC receptacles 27a, 230 VAC receptacles 27c, and 12 V DC receptacles 27b, or any combination thereof. The system 100 may be configured to use an onboard battery when not operably connected to the alternator of the armored truck. Alternatively, the system 100 may comprise no battery such that the system 100 only provides power to the charging pins 11 and UV emitters 15 when plugged into a stationary power outlet, such as a wall outlet.

In the preferred embodiment, the system 100 may comprise multiple power supplies. For instance, the system 100 may be optimized to receive power from a battery and a second power source, such as a wall outlet or alternator. A control board of the system 100 may cause the second power source to charge the batteries of the system 100 while concurrently providing power to the charging pins 11 and UV emitters 15. Once the batteries have been charged, the control board may be configured to cause the second power source to cease charging the batteries while continuing to supply the system 100 with power. The control board may also break a circuit between the batteries and the various components of the system 100 to prevent the batteries from losing power once fully charged and still connected to the secondary power source. Once the system 100 is disconnected from the second power source, the control board may cause the system 100 to be powered using the batteries, allowing the system 100 to operate in situations where mobility is desired.

In some preferred embodiments, the circuitry 600 of the system 100 may require an additional circuit piece to complete circuits between the power supply 30 and the charging pins 11 and UV emitters 15. At least one switch 34, 35, illustrated in FIG. 6, may be used to complete the circuits within the system 100, thus allowing the transfer of power from the power supply 30 to the UV emitters 15 and charging pins 11 when certain conditions are met. In one preferred embodiment, an interlock switch 34 may be connected to the circuitry 600 in a way such that closing the door 22 of the vessel 10 causes the interlock switch 34 to close a circuit connecting the UV emitters 15 and/or charging pins 11 to the power supply 30, thus allowing the sanitizing containers to receive power and/or allowing the UV emitters 15 to sanitize the contents of the vessel 10. In another preferred embodiment, a switch may be configured in a way such that a user may open and close the circuit manually regardless of the position of the door. This may allow a user to manually control the power transferred to the charging pins 11 and/or UV emitters 15 without the need for opening or closing the door 22. In yet another preferred embodiment, a switch 35 may be attached to the circuitry connecting a geolocation device and/or communication repeater 33 of the system 100 to the power supply, which may be used to stop the transmission of geospatial data by shutting of the power. In one preferred embodiment, the switch may be configured to transmit a signal to the sanitizing containers in a way such that the control boards of the sanitizing containers may stop transmitting geospatial data. This may allow the charging assembly and/or sanitizing containers to be EMI/RFI secure in order to prevent tracking when a user determines a threat is present.

The geolocation device may be a single component of a larger computing device, such as the Global Positioning System (GPS) receiver module. In a preferred embodiment, the geolocation device is located within the volume of at least one of the top or bottom. In one preferred embodiment, the geolocation device may comprise a plurality of devices working together to obtain a geolocation via triangulation. In a preferred embodiment, the geolocation device is a GPS sensor. The GPS sensor may measure and transmit geospatial data relevant for determining geolocation. A GPS sensor may be defined as a receiver having an antenna designed to communicate with a navigation satellite system. Geospatial data may be spatial data including, but not limited to, numeric data, vector data, and raster data, or any combination thereof. Numeric data may be statistical data which includes a geographical component or field that can be joined with vector files so the data may be queried and displayed as a layer on a map in a GIS. Vector data may be data that has a spatial component, or X, Y coordinates assigned to it. Vector data may contain sets of points, lines, or polygons that are referenced in a geographic space. Raster data may be data in a .JPG, .TIF, .GIF or other picture file format. For instance, a map scanned in a flatbed scanner may be considered raster data. A raster dataset may comprise a number of cells, wherein every cell within the plurality of cells of the raster dataset belongs to at least one zone. Each group of connected cells in a zone is considered a region. A zone that consists of a single group of connected cells may have only one region, and the number of cells that make up a region has no practical limit. Zones may be composed of as many regions as necessary to represent a map feature.

As illustrated in FIGS. 1 and 3, the preferred embodiment of the charging assembly may comprise UV emitter within the cavity of the vessel 10, which are used to sanitize items therein. In a preferred embodiment, the UV radiation emitted by the UV emitters 15 is generally between 200 and 300 nanometers; however, other wavelengths may be desirable depending on the bacteria/virus desired to be eradicated. In some preferred embodiments, the system 100 may further comprise light defusing waveguides, which may provide the internal cavity of the vessel 10 with uniform and complete UV radiation. Types of devices that may act as waveguides include, but are not limited to, strip waveguides, rib waveguides, segmented waveguides, laser-inscribed waveguides, and optical fiber, or any combination thereof. In the preferred embodiment, the UV emitters 15 are located on and/or alongside the charging rails and are situated such that UV light is directed to the sides of a sanitizing container. In some preferred embodiments, UV intensity sensors 32 of the UV monitor 29 may be used to measure UV intensity within the internal cavity of the vessel 10. When the system 100 detects that the amount of UV radiation within the vessel 10 does not reach a threshold required to sanitize the contents within, the system 100 may cause an indicator light 17, 18, 19 to be illuminated, which may instruct a user of a possible fault in the circuity and/or indicate to the user that a defective UV emitter needs replacing. In another preferred embodiment, the system may comprise a timed circuit 16 that may switch off power to the UV emitters after a specified period of time.

Some preferred embodiments of the system 100 may comprise at least one indicator light 17, 18, 19, which may be used to alert a user when the system's 100 communication repeater is in operation. Meters 20 of the system 100 may be used to indicate the amount of charge and output voltage a battery of the charging assembly is undergoing. Meters 20 may also be used to indicate the amount of UV radiation there is within the cavity. In one preferred embodiment, an indicator light 17, 18, 19 may be used to indicate when the charging pins 11 and/or UV emitters 15 are receiving power, which may act as a warning to a user who does not want to access the internal cavity while the charging pins 11 are receiving power and/or the UV emitters 15 are emitting UV radiation. Some preferred embodiments of the system 100 may comprise a display used to indicate the environment of the internal cavity of the vessel 10. A display may be defined as an output device that communicates data that may include, but is not limited to, visual, auditory, cutaneous, or any combination thereof. Displays may include, but are not limited to, LCD monitors, light emitting diode (LED) monitors, gas plasma monitors, screen readers, speech synthesizers, speakers, or any combination thereof, but is not limited to these devices.

FIG. 7 provides a flow chart 700 illustrating certain, preferred method steps that may be used to carry out the method of sanitizing containers using the charging assembly. Step 705 indicates the beginning of the method. During step 710, a user may obtain a charging assembly and subsequently obtain a sanitizing container during step 715. Once the charging assembly and sanitizing container have been obtained, the user may open the charging assembly during step 720. The user may then perform a query to determine whether to connect the sanitizing container to the charging pins 11 of the charging assembly during step 725. Based on the results of the query, the user may take an action during step 730. If the user determines that the container should be connected to the charging pins 11, the user may attach the sanitizing container during step 732. If the user determines the sanitizing container should not be attached to the charging pins 11, the user may place the wire racks 13 of the system 100 during step 733. Once the sanitizing container has been placed within the charging assembly, the user may close the door 22 during step 735.

A user may then determine for what purpose the charging assembly is being used by performing a query during step 740, which may instruct a user in how to proceed. Based on the results of the query, the user may take an action during step 745. If the user determines that the charging assembly is only being used to sanitize the contents within the internal cavity or only being used to transport contents from one location to another, the user may maintain UV exposure within the cavity for a preset amount of time during step 747 and subsequently remove the sanitizing containers during step 752. The user may then proceed to terminate method step 755. If the user determines that the charging assembly is being used for charging purposes, the user may wait for the sanitizing containers to fully charge during step 748 and subsequently remove the sanitizing containers during step 752 before proceeding to the terminate method step 755.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, materials, and arrangements of the parts and process stages which have been described and illustrated in order to explain the nature of this inventive subject matter can be made without departing from the principles and scope of the inventive subject matter.

What is claimed is:

1) A charging assembly comprising:
a vessel having at least two sides, bottom, top, and door,
wherein said top is connected to said at least two sides at a top end of said at least two sides,
wherein said bottom is connected to said at least two sides at a bottom end of said at least two sides,
wherein an internal cavity is created by said at least two sides, top, bottom, and door,
a sanitizing container configured to hold currency therein,
wherein said sanitizing container is configured to heat an internal cavity in a way such that said currency is sanitized,
a charging pin operably connected to a power supply,
wherein said charging pin is configured to provide power to said sanitizing container via a charging port,
a spacing rod configured to support a plurality of containers in a way that reduces movement,
wherein said spacing rod is offset from said charging pin, and
a UV emitter operably connected to a power supply,
wherein said UV emitter is configured to emit UV light between 200 and 300 nanometers.

2) The charging assembly of claim 1, further comprising a locking device secured on said bottom end of said vessel,
wherein said locking device comprises an engine operably connected to a locking element via a tow,
wherein said tow is configured to spool and unspool about a drum of said engine,
wherein said drum is configured to spool and unspool said tow based on an action of said engine.

3) The charging assembly of claim 2, further comprising a vehicle having a stationary fastener on a base surface,
wherein said locking element of said locking device is configured to secure to said stationary fastener,
wherein said locking device creates a downward force that acts on said vessel by pulling said bottom end of said vessel towards said base surface when secured to said stationary fastener,
wherein said downward force keeps said vessel stationary when said vehicle is in motion.

4) The charging assembly of claim 3, wherein said charging pin is operably connected to said power supply of said vehicle.

5) The charging assembly of claim 4, wherein a switch of said vehicle allows a user to control a supply of power transferred from said power supply to said UV emitter.

6) The charging assembly of claim 5, wherein a light operably connected to said switch is configured to emit light when said UV emitter receives power from said power supply.

7) The charging assembly of claim 1, further comprising a geolocation device configured to transmit geospatial data concerning a geolocation of said vessel.

8) The charging assembly of claim 1, further comprising wheels secured to said bottom of said vessel.

9) The charging assembly of claim 8, wherein said wheels are spring-loaded and configured to compress when a downward force is applied to said vessel.

10) The charging assembly of claim 1, further comprising a volume within at least one of said bottom and said top,
wherein said volume is configured to contain circuitry therein.

11) The charging assembly of claim 1, wherein a seal connected to said at least two sides, top, and bottom allow said door to seal the internal cavity of said vessel.

12) A charging assembly comprising:
a vessel having at least two sides, bottom, top, and door,
wherein said top is connected to said at least two sides at a top end of said at least two sides,
wherein said bottom is connected to said at least two sides at a bottom end of said at least two sides,
wherein an internal cavity is created by said at least two sides, top, bottom, and door,
a charging pin operably connected to a power supply,
wherein said charging pin is configured to provide power to a charging port,
a spacing rod configured to support a plurality of containers in a way that reduces movement,
a UV emitter operably connected to a power supply,
wherein said UV emitter is configured to emit UV light between 200 and 300 nanometers,
wherein said UV emitter is positioned within said internal cavity in a way such that said UV light illuminates said plurality of containers, and
a locking device secured on said bottom end of said vessel,
wherein a locking element of said locking device is configured to secure to a stationary fastener,
wherein said locking device creates a downward force that acts on said vessel by pulling said bottom end of said vessel towards said stationary fastener when secured to said stationary fastener.

13) The charging assembly of claim 12, further comprising a vehicle having said stationary fastener secured to a base surface,
wherein said locking element of said locking device is configured to secure to said stationary fastener of said vehicle,
wherein said downward force keeps said vessel stationary when said vehicle is in motion.

14) The charging assembly of claim 13, wherein a switch of said vehicle allows a user to control a supply of power transferred from said power supply to said UV emitter.

15) The charging assembly of claim 14, wherein a light operably connected to said switch is configured to emit light when said UV emitter receives power from said power supply.

16) The charging assembly of claim 12, further comprising a geolocation device configured to transmit geospatial data concerning a geolocation of said vessel.

17) The charging assembly of claim 12, further comprising wheels secured to said bottom of said vessel,
wherein said wheels are spring-loaded and configured to compress when a downward force is applied to said vessel.

18) The charging assembly of claim 12, wherein a seal connected to said at least two sides, top, and bottom allow said door to seal the internal cavity of said vessel.

19) A method for charging a sanitizing container comprising steps of:
obtaining a charging assembly,
wherein said charging assembly is configured to hold a plurality of sanitizing containers,
obtaining a sanitizing container of said plurality of sanitizing containers,
wherein said sanitizing container is configured to sanitize currency via at least one of heat and UV light,
placing said sanitizing container within an internal cavity of said charging assembly,
attaching said sanitizing container to a charging pin of said charging assembly,
closing a door of said charging assembly,
wherein closing said door causes a switch of said charging assembly to create a closed circuit,
wherein said closed circuit allows a supply of power to be transferred from a power supply to said charging pin,
wherein said closed circuit allows said supply of power to be transferred from said power supply to a UV emitter, and
waiting for UV light of said UV emitter to sanitize an exterior surface of said sanitizing container.

20) The method of claim 19, further comprising the steps of:
waiting for a battery of said sanitizing container to charge,
opening said door of said charging assembly once at least one of said exterior surface is sanitized and said battery is charged,
wherein opening said door causes said switch of said charging assembly to create an open circuit,
wherein said open circuit does not allow said supply of power to be transferred from said power supply to said charging pin,
wherein said open circuit does not allow said supply of power to be transferred from said power supply to said UV emitter, and
removing said sanitizing container from said charging assembly.
